(19) United States
(12) Patent Application Publication
(10) Pub. No.: US 2017/0035863 A1
(43) Pub. Date: Feb. 9, 2017

(54) Compositions Comprising Variegin

(71) Applicant: UNIVERSITY OF LEEDS, Leeds (GB)

(72) Inventor: Helen PHILIPPOU, Leeds (GB)

(73) Assignee: UNIVERSITY OF LEEDS, Leeds (GB)

(21) Appl. No.: 15/304,451

(22) PCT Filed: Apr. 16, 2015
(86) PCT No.: PCT/GB2015/051150

§ 371 Date: Oct. 14, 2016

(30) Foreign Application Priority Data
Apr. 16, 2014 (GB) 1406863.9

(51) Int. Cl. A61K 38/57; A61K 45/06; A61K 9/00

(52) U.S. Cl. CPC A61K 38/57; A61K 9/0019; A61K 45/06

(57) ABSTRACT

There is described variegin for use as a medicament in the treatment of disease or condition characterised in that the variegin is administered in an amount of at least about 0.1 mg/kg (mass of drug compared to mass of patient).

### FIELD OF THE INVENTION

The present invention relates to novel compositions, to novel methods of treatment and novel methods of delivering therapeutically active agents.

More particularly, the invention relates to novel compositions comprising a high dose regime of variegin, and variant peptides thereof, said compositions are suitable as anticoagulants which demonstrate a lack of bleeding risk to patients.

### BACKGROUND TO THE INVENTION

Thrombosis is a common cause of mortality. Anticoagulants are employed to treat and prevent thrombosis. However, treatment with anticoagulants presents a major clinical challenge because of the high incidence of major bleeding associated with their use (1-3%). Even new anticoagulants, recently approved, exhibit a high incidence of bleeding. This is of particular concern during and after surgery when the risk of bleeding has to be balanced against the need to provide protection against deep venous thrombosis and pulmonary embolism (venous thromboembolism).

Thromboprophylaxis is the treatment given to prevent blood clots occurring during/following a joint replacement. In 2010, 76,759 UK patients were recorded by the National Joint Registry (NJR) as having hip replacement surgery, a 6% increase on 2009. Of these patients, 68,907 were primary replacements and the remaining 11% were revisions. In 2011, 81,979 patients undergoing knee replacement procedures were recorded by NJR, a 5.7% increase from 2010. Of these patients, 76,870 were primary procedures and the remainder were revisions. At present heparin or its low moleculare weight (LMWH) derivatives is the drug of choice for thromboprophylaxis, commonly used with elasticated stockings. In 2007, 63% of all patients undergoing replacement surgery were prescribed both elasticated stockings and heparin; by 2010 this had risen to 87% of all patients.

All current clinically used anticoagulants exhibit a risk of bleeding.

International Patent application No. WO2003/091284, Evolutec, describes naturally occurring, anticoagulants derived from the salivary glands of haematophagous arthropods. In particular, WO '284 describes “EV445 protein” which was isolated from the salivary glands of the tick Amblyomma variegatum (tropical bont tick), which was found to be an anticoagulant and a thrombin inhibitor. “EV445 protein” is otherwise known as variegin. Variegin possesses some similarities to hirudin, a naturally occurring anticoagulant, isolated from the salivary glands of medicinal leeches (such as Hirudo medicinalis) and marketed as bivalirudin.

Uncontrolled activation of the coagulant system or defective inhibition of the activation processes may cause formation of local thrombi or embolisms in vessels (arteries, veins, lymph vessels) or in heart cavities. This may lead to serious disorders, such as myocardial infarction, angina pectoris, reocclusions and restenoses after angioplasty or aortocoronary bypass, stroke, transitory ischaemic attacks, peripheral arterial occlusive disorders, pulmonary embolisms or deep venous thromboses.

International Patent application No. WO2008/155658, IZSAS, describes synthetic variegin and derivatives thereof as anti-coagulants. Describes an effective dose will be from 0.01 mg/kg (mass of drug compared to mass of patient) to 50 mg/kg.

International Patent application No. WO2010/128285, NERC, describes a method of producing a modified serine protease inhibitor (SPI) displaying the unique functionally of variegin, by introducing one or more amino acids comprising a methionine-histidine-lysine or methionine-histidine-arginine sequence into the SPI.

However, more recently, Capodanno D., et al in a review article “Bivalirudin for acute coronary syndromes: premises, promises and doubts” Thrombosis and Haemostasis 113.4/2015 describes that whilst bivalirudin is a valuable anticoagulant option in patients with acute coronary syndromes (ACS) undergoing percutaneous coronary intervention; clinical evidence supporting the use of bivalirudin over heparin in current ACS guidelines, that no longer reflect current management patterns, now including the use of oral antiplatelet agents more potent than clopidogrel (i.e. prasugrel or ticagrelor) and a broader implementation of strategies to reduce bleeding (i.e. radial access for percutaneous coronary intervention, and use of glycoprotein IIb/IIIa inhibitors only in bailout situations). Capodanno reports that in clinical practice it remains a challenge to define the balance between bivalirudin efficacy and safety over heparins in the context of other antithrombotic treatments.

Furthermore, Journal of the American Medical Association, Apr. 7, 2015 Volume 313, Number 13, 1323-1324, Cavender et al report that bivalirudin has been studied as an alternative to heparin for patients undergoing percutaneous coronary intervention (PCI) with stable coronary artery disease, non-ST-segment elevation acute coronary syndrome (NSTE-ACS), and ST-elevation myocardial infarction (STEMI). These studies found that bivalirudin reduced major bleeding when compared with regimens of heparin plus glycoprotein IIb/IIIa (Gp IIb/IIIa) inhibitors. However, many of these trials also found small numerical increases in ischemic events with bivalirudin and increases in acute stent thrombosis, particularly in patients with STEMI.

The data are now quite consistent in that bivalirudin is less efficacious than heparin, particularly when it comes to stent thrombosis within the first 3 hours after PCI. Continuing the infusion 3 hours post-PCI is an expensive partial solution to the stent thrombosis problem. In addition, because bivalirudin is cleared by the kidneys, bivalirudin use is also problematic in the increasingly prevalent population of patients with renal impairment. Thus, the use of bivalirudin has many limitations.

The present invention has identified that a high therapeutic dose of variegin e.g. 20 mg/kg or more, does not cause bleeding. By comparison, a high dose of known anticoagulants currently in use, LMWH, causes bleed-out. However, the finding that variegin, at a high therapeutic dose, does not cause bleeding, is not obvious to a person skilled in the art.

### SUMMARY TO THE INVENTION

The present invention provides a peptide anticoagulant that is able to provide significant inhibition of thrombus formation (≧50% inhibition) without any potential risk of bleeding; demonstrated by no prolongation of bleeding time or increased blood loss versus controls in a murine model of thrombosis.

In contrast at the dose of LMWH required to achieve 50% inhibition of thrombus formation, the bleeding time was prolonged by >5-fold in the same model of thrombosis.

Variegin is a direct inhibitor of thrombin that has an unprecedented anticoagulant effect in vivo with no associated risk of bleeding at doses achieving 50% inhibition of fibrin clot formation. In comparative studies, LMWH at a therapeutic dose (200 IU/kg) administered intravenously to mice only resulted in 22% inhibition of clot growth with a 69 second prolongation of bleeding time and 1.3-fold increase in blood loss compared with controls. Increasing the dose of LMWH to 4,000 IU/kg only inhibited clot growth by ˜50% with a >5-fold prolongation of bleeding time to >1800s compared to 360s for controls and >14-fold increased blood loss compared to controls. However, in the same study, variegin and variants thereof, at 20 mg/kg inhibited >50% clot growth with no prolongation of bleeding time or any major increase in blood loss (except SYM-3871 that required a dose of 40 mg/kg to achieve 50% inhibition of clot formation; and at 20 mg/kg of SYM-3491-SO3 showed ˜70% inhibition at the 33 minute time frame with an approximate 2-fold prolongation of bleeding time at the end of the experiment).

The risk of bleeding on administration of a thrombin inhibitor generally restricts the dose of thrombin inhibitor available to administer to a patient. However, the absence of any potential risk of bleeding with the use of variegin has hitherto been unknown.

Thus, according to a first aspect of the invention there is provided variegin for use as a medicament in the treatment of disease or condition characterised in that the variegin is administered in an amount of at least about 0.1 mg/kg (mass of drug compared to mass of patient). The disease or condition which may be treated according to this aspect of the invention is as hereinafter defined.

According to another aspect of the invention there is provided the use of variegin in the manufacture of a medicament in the treatment of disease or condition wherein the amount of variegin is at least about 0.1 mg/kg (mass of drug compared to mass of patient).

According to a yet further aspect of the invention there is provided the use of variegin in the manufacture of a medicament in the treatment of disease or condition wherein the amount of variegin administered to a patient is sufficient to achieve a plasma concentration of variegin of from about 1 ng/ml to about 1.5 g/L (based on a 100 mg/kg dose).

Alternatively, the amount of variegin administered to a patient may be sufficient to achieve at least 40% anticoagulation with concomitant minimal bleeding risk.

In the use according to this aspect of the invention the amount of variegin may be sufficient to achieve a plasma concentration of at least 25 pM of variegin and is maintained for at least 2 hours in the patient. It will be understood by the person skilled in the art that this should be considered to be the lowest desirable variegin plasma concentration, therefore, the use of higher plasma concentrations is contemplated within the scope of the present invention.

Variegin is a tick-derived 32 residue protein having the amino acid sequence first described in WO03/091284, which is hereinbefore described and which is incorporated herein by reference.

The uses and methods described herein may be performed using variegin that is obtained by any means, for example, natural or synthetic variegin. In addition references to variegin used herein shall, unless otherwise stated, also refer to variants of variegin, for example, those described in International Patent application No. WO2010/128285, which is incorporated herein by reference. Particular peptide variants of variegin shall include, but shall not be limited to, SYM-3871, SYM-3870-S03 and SYM-3491-SO3, as defined herein. Reference to “variegin only” shall mean variegin, not including variants of variegin.

Accordingly, variegin of the invention may be produced using any methodology known in the art, e.g., chemical peptide synthesis, solid-phase or solution-phase peptide synthesis, in vitro translation from a nucleic acid molecule encoding a modified SPI, or cell-based production methods employing prokaryotic or eukaryotic recombinant expression systems.

The patient is generally a mammal such as a human, cow, sheep, pig, camel, horse, dog, cat, monkey, mouse, rat, hamster, rabbit and the like.

The dosage of at least about 10 mg/kg (mass of drug compared to mass of patient) of variegin may be from about 10 mg/kg to about 100 mg/kg and may comprise a therapeutically effective amount, i.e. the amount of compound needed to treat or ameliorate a disease or condition; or a prophylactically effective amount, i.e. an amount of compound needed to prevent a targeted disease or condition. Alternatively, the dosage of variegin may be at least about 20 mg/kg (mass of drug compared to mass of patient). The exact dosage will generally be dependent on the status of the patient at the time of administration. Factors that may be taken into consideration when determining dosage include the severity of the disease state in the subject, the degree of anticoagulation required, the general health of the subject, the age, weight, gender, pregnancy, diet, time and frequency of administration, drug combinations, reaction sensitivities and the subject's tolerance or response to therapy.

The precise amount can be determined by routine experimentation, but may ultimately lie with the judgement of the clinician or veterinarian. Generally, an effective dose, i.e. a therapeutically effective dose or a prophylactically effective dose will be from about 10 mg/kg to about 100 mg/kg; from about 20 mg/kg to about 100 mg/kg; or about 30 mg/kg to about 100 mg/kg; or about 40 mg/kg to about 100 mg/kg; or about 50 mg/kg to about 100 mg/kg; or about 60 mg/kg to about 100 mg/kg; or about 70 mg/kg to about 100 mg/kg; or about 80 mg/kg to about 100 mg/kg; or about 90 mg/kg to about 100 mg/kg. Preferably the amount of variegin present is about >50 mg/kg. Alternatively, the amount of variegin may be from about >50 mg/kg to about 100 mg/kg, or from about >50 mg/kg to about 90 mg/kg, or from about >50 mg/kg to about 80 mg/kg, or from about >50 mg/kg to about 75 mg/kg, or from about >50 mg/kg to about 70 mg/kg, or from about >50 mg/kg to about 60 mg/kg, for example, from about 51 mg/kg to about 60 mg/kg, or from about 55 mg/kg to about 60 mg/kg.

A therapeutically effective amount or suitable dose of variegin may be determined by comparing its in vitro activity and in vivo activity in an animal model. Methods for extrapolation of effective dosages in mice and other test animals to humans are known. The precise dose will depend upon a number of factors, including, inter alia, whether the use of variegin is for prevention or for treatment, the size and location of the area to be treated, the precise nature of the variegin.

The variegin of the invention may be provided in the form of a pharmaceutical composition in conjunction with a pharmaceutically acceptable carrier.

Thus, according to this aspect of the invention there is provided a pharmaceutical composition comprising variegin in conjunction with a pharmaceutically acceptable carrier wherein the amount of variegin is at least about 10 mg/kg (mass of drug compared to mass of patient). Alternatively, the pharmaceutical composition may comprise an amount of variegin of at least about 20 mg/kg (mass of drug compared to mass of patient).

In the pharmaceutical composition according to this aspect of the invention the amount of variegin present may be sufficient to achieve a plasma concentration of variegin in a patient of from about lng/ml to about 1.5 g/L.

The term “pharmaceutically acceptable carrier” will be understood by the person skilled in the art and includes, inter alia, genes, polypeptides, antibodies, liposomes, polysaccharides, polylactic acids, polyglycolic acids and inactive virus particles or indeed any other agent provided that the excipient does not itself induce toxicity effects or cause the production of antibodies that are harmful to the individual receiving the pharmaceutical composition.

Pharmaceutically acceptable carriers may additionally contain liquids such as water, saline, glycerol, ethanol or auxiliary substances such as wetting or emulsifying agents, pH buffering substances and the like. Excipients may enable the pharmaceutical compositions to be formulated into tablets, pills, dragees, capsules, liquids, gels, syrups, slurries, suspensions to aid intake by the subject. A thorough discussion of pharmaceutically acceptable carriers is available in Remington's Pharmaceutical Sciences (Mack Pub. Co., N.J. 1991).

In another aspect of the present invention there is provided a method of treating a patient suffering from an increased risk of thrombosis or preventing a subject developing a thrombotic event, said method comprising administering variegin to the patient in an amount of at least about 0.1 mg/kg (mass of drug compared to mass of patient).

By “increased risk of thrombosis” is meant any circumstance that will generate an increased risk of thrombotic tendency following stimulation of coagulation system and/or platelet activation (for example during surgical procedures) or by hereditary conditions such as thrombophilias).

Variegin has applications in the treatment and prevention of a wide range of diseases and conditions and may be used in any situation in which it is desired to induce anticoagulation to prevent or treat increased risk of thrombosis.

Treatment when anticoagulation is desirable includes, but shall not be limited to, procedures involving percutaneous, transvascular or transorgan catheterisation, coronary angioplasty; endo-vascular stent procedures; direct administration of thrombolytic agents via an arterial or venous catheter such as following stroke or coronary thrombosis; electrical cardioversion; placement of cardiac pacemaker leads; intravascular and intracardiac monitoring of pressure, gaseous saturation or other diagnostic parameters; radiological and other procedures involving percutaneous or transorgan catheterisation; to ensure the patency of long-term, indwelling, intravascular parenteral nutritional catheters; to ensure the patency of vascular access ports whether long or short term, and orthopaedic surgery.

At the dose of at least about 0.1 mg/kg (mass of drug compared to mass of patient) of variegin it is anticipated that the incidence of perioperative or postoperative bleeding will be substantially reduced and in patients with acute coronary syndrome the incidence of subsequent myocardial infarction will be reduced. Therefore, the use of variegin as hereinbefore described will also be superior to heparin and its analogues for use during such procedures.

Additional in vivo applications of the methods of the first aspect of the invention include emergency anticoagulation after a thromboembolic event including but not limited to: acute myocardial infarction; thrombotic stroke; deep venous thrombosis; thrombophlebitis; pulmonary embolism; embolic and micro-embolic episodes where the source of the episodes may be the heart, atherosclerotic plaque, valvular or vascular prostheses or an unknown source; disseminated intravascular coagulation (DIC).

Anticoagulation may also be desirable during surgical procedures. The high risk of venous thromboembolism among patients undergoing lower limb surgery, e.g. hip or knee replacement, is well recognised and guidelines for the appropriate prophylaxis have been developed. However, because of the risk of postoperative bleeding, there have been concerns amongst orthopaedic surgeons about the prophylactic use of anticoagulant therapy. Due, inter alia, to the demonstrated absence of bleeding time or increased blood loss, it would be readily understood by the person skilled in the art that a dose of at least about 0.1 mg/kg of variegin will be suitable for post-operative administration to a patient.

Inhibition of thrombin by variegin as hereinbefore described may be of clinical benefit in the treatment of any thrombin-mediated condition. A thrombin-mediated condition may include disorders associated with the formation or activity of thrombin.

Thrombin plays a key role in haemostasis, coagulation and thrombosis. Thrombin-mediated conditions include thrombotic conditions, such as thrombosis and embolism. Thrombosis is coagulation which is in excess of what is required for haemostasis (i.e. excessive coagulation), or which is not required for haemostasis (i.e. extra-haemostatic or non-haemostatic coagulation).

Thrombosis is blood clotting within the blood vessel lumen. It is characterised by the formation of a clot (thrombus) that is in excess of requirement or not required for haemostasis. The clot may impede blood flow through the blood vessel leading to medical complications. A clot may break away from its site of formation, leading to embolism elsewhere in the circulatory system. In the arterial system, thrombosis is typically the result of atherosclerotic plaque rupture.

In some embodiments, thrombosis may occur after an initial physiological haemostatic response, for example damage to endothelial cells in a blood vessel. In other embodiments, thrombosis may occur in the absence of any physiological haemostatic response.

Thrombosis may occur in individuals with an intrinsic tendency to thrombosis (i.e. thrombophilia) or in “normal” individuals with no intrinsic tendency to thrombosis, for example in response to an extrinsic stimulus.

Thrombosis and embolism may occur in any vein, artery or other blood vessel within the circulatory system and may include microvascular thrombosis.

Thrombosis and embolism may be associated with surgery (either during surgery or afterwards) or the insertion of foreign objects, such as coronary stents, into a patient. For example, variegin as described herein may be useful in the surgical and other procedures in which blood is exposed to artificial surfaces, such as open heart surgery, extracorporeal membrane oxygenation (ECMO) and dialysis. Thrombotic conditions may include thrombophilia, thrombotic stroke, coronary artery occlusion and venous thrombosis.

Patients suitable for treatment as described herein include patients with conditions in which thrombosis is a symptom or a side-effect of treatment or which confer an increased risk of thrombosis or patients who are predisposed to or at increased risk of thrombosis, relative to the general population. For example, variegin as described herein may also be useful in the treatment or prevention of venous thrombosis in cancer patients, and in the treatment or prevention of hospital-acquired thrombosis, which is responsible for 50% of cases of venous thromboembolism. Variegin may exert a therapeutic or other beneficial effect on thrombin-mediated conditions, such as thrombotic conditions, without substantially inhibiting or impeding haemostasis. For example, the risk of haemorrhage in patients treated with variegin may not be increased or substantially increased relative to untreated individuals.

Individuals treated with conventional anticoagulants, such as natural and synthetic heparins, warfarin, direct serine protease inhibitors (e.g. argatroban, dabigatran, apixaban, and rivaroxaban), hirudin and its derivatives (e.g. lepirudin and bivalirudin), and anti-platelet drugs (e.g. clopidogrel, ticlopidine and abciximab) cause bleeding. The risk of bleeding in patients treated with variegin may be reduced relative to individuals treated with conventional anticoagulants.

Thrombin-mediated conditions include non-thrombotic conditions associated with thrombin activity, including inflammation, infection, tumour growth and metastasis, organ rejection and dementia (vascular and non-vascular, e.g. Alzheimer's disease).

Particular patient groups include those patients who are considered to be at greater risk of bleeding, for example, those requiring dose escalation e.g. obese, kidney diseases, liver disease, iv drug users, pregnancy, paediatric, geriatric, cancer, extended-duration prophylaxis of venous thromboembolism (VTE) in acute medically ill patients, breastfeeding, percutaneous coronary intervention, orthopaedic surgery, abdominal aortic aneurysm (AAA), etc. In addition, patient groups include those patients requiring additional platelet inhibition with anticoagulation, such as, percutaneous coronary intervention (PCI); patients on thrombolytic therapy (tissue plasminogen activator, (tPA)) for treatment of ischaemic stroke; or patients requiring cardiopulmonary bypass surgery or extracorporeal membrane oxygenation (ECMO) and dialysis.

Currently, heparin is used in coatings to reduce the risk of complications due to fibrin deposition, which is an important cause of patient morbidity. Thus, it is within the scope of the present invention to provide a medical device, such as a catheter, stent, orthopaedic implant, etc. coated with variegin or a pharmaceutical composition comprising variegin as hereinbefore described.

Additionally, the use and the methods of the present invention may be useful to induce anticoagulation in heparin-resistant patients.

In the case of the administration of relatively large doses of variegin, it may be advisable to divide these over the course of the day, namely into several individual doses or as a continuous infusion or as a sustained release formulation.

The pharmaceutical compositions according to the invention can be used for the treatment of the above-mentioned indications when they are administered parenterally, such as, intravenous, intramuscular or subcutaneous injection, or enterally, such as, oral administration.

There are suitable infusion or injection solutions, preferably aqueous isotonic solutions or suspensions, it being possible to prepare these before use, for example from lyophilised preparations that contain the active ingredient(s) alone or together with a pharmaceutically acceptable carrier, such as mannitol, lactose, dextrose, human serum albumin and the like. The pharmaceutical compositions are sterilized and, if desired, mixed with adjuncts, for example preservatives, stabilisers, emulsifiers, solubilisers, buffers and/or salts (such as 0.9% sodium chloride) for adjusting the isotonicity. Sterilization can be achieved by sterile filtration through filters of small pore size (0.45 μm diameter or smaller) after which the composition can be lyophilised, if desired. Antibiotics may also be added in order to assist in preserving sterility.

Variegin may be administered individually to a patient or may be administered in combination with other pharmaceutically active agents, for example, with other anticoagulants.

The combination compositions of this aspect of the invention may include, inter alia, conventional anticoagulants, such as, tissue plasminogen activator (tPA), natural and synthetic heparins, warfarin, direct serine protease inhibitors (e.g. argatroban, dabigatran, apixaban, and rivaroxaban), hirudin and its derivatives (e.g. lepirudin and bivalirudin), and anti-platelet drugs (e.g. clopidogrel, ticlopidine and abciximab). Combination compositions which may be of particular interest are combinations of variegin with tPA or with anti-platelet drugs, such as, clopidogrel, ticlopidine and abciximab. The risk of bleeding in patients treated with conventional anticoagulants may be reduced by co-administration with variegin as herein before described. Such combination compositions may be novel per se and according to the invention can be used in mammals (humans or animals) for the prevention or treatment of thrombosis or diseases caused by thrombosis, arteriosclerosis, myocardial and cerebral infarction, venous thrombosis, thromboembolism, post-surgical thrombosis, thrombophlebitis, etc.

Use of variegin in a combination therapy with tPA may be particularly useful in the treatment of certain disorders, including, inter alia, ischemic stroke, myocardial infarction, and the like.

The method of treating a patient as hereinbefore described may also comprise the administration of an aforementioned combination composition. Alternatively, the method of treatment may comprise the administration of variegin in combination with conventional anticoagulants, separately, simultaneously or sequentially.

Such combination compositions according to this aspect of the invention may be in a state to allow the active ingredients (including variegin) to be administered (e.g. infused) at the same time and by the same route (i.e. cannula) or to apply, for example, variegin first, e.g. by bolus injection, and then, starting within 5 to 10 minutes thereafter, the second or combination active agent.

Another advantage of the reduced bleeding risk is that variegin is suitable for use in a sustained release formulation. Sustained release formulations are generally designed to slowly release the active agent (variegin) from the delivery device, e.g. a tablet or capsule. The sustained or slow release can prolong blood levels of the active agent and with variegin the side effect of bleeding is minimised. Sustained release formulations will often be oral delivery forms. Examples of such oral sustained release delivery forms include, but shall not be limited to, tablets or capsules.

One example of a sustained release oral composition comprising variegin is a tablet core comprising a therapeutically effective amount of variegin, a water swellable polymer, optionally a neutralizing agent, and a substantially water insoluble film coating surrounding the tablet core.

Such sustained release oral compositions comprising variegin are novel per se. Therefore, according to this aspect of the invention the active ingredient, variegin, may be present in the tablet core in an amount of from about 10% and about 60% by weight of the total core mass.

The tablet core may typically be in the form of a solid conventional tablet. Generally, the core is compressed into its final shape using a standard tablet compressing machine. Thus, the core may contain compressing aids and diluents such as lactose that assist in the production of compressed tablets. The core can be comprised of a mixture of agents combined to give the desired manufacturing and delivery characteristics.

The term “water swellable polymer” generally refers to a polymer used in the tablet core that is capable of swelling upon hydration. The term “swellable” implies that the polymer is in a non-hydrated state. Examples of water swellable polymers include, but shall not be limited to, acrylate polymers, Carbopol™ polymers, and the like.

When present, a “neutralizing agent” acts to modulate hydration of the swellable polymer and provides for release of the active ingredient from the tablet core into the gastrointestinal tract by diffusion directly from the core and by extrusion of swelling polymer. The neutralizing agent is solubilized by the aqueous media of the environment and establishes an environment such that the environment pH is appropriate for the desired polymer gel particle hydration rate, for example, by neutralization of acidic functional groups on the polymer, thereby affecting the hydration rate.

Compounds that can suitably be used as neutralising agents include, but shall not be limited to, bases and salts thereof, such as, sodium carbonate, sodium bicarbonate, sodium citrate, arginine, meglamine, sodium acetate, sodium phosphates (e.g., sodium phosphate dibasic anhydrous), potassium phosphates, calcium phosphate, ammonium phosphate, magnesium oxide, magnesium hydroxide, sodium tartrate and the like.

Other compounds that can be used as polymer hydration modifiers include sugars such as lactose, sucrose, mannitol, sorbitol, pentaerythritol, glucose and dextrose.

Polymers such as microcrystalline cellulose and polyethylene glycol, as well as surfactants and other organic and inorganic salts can also be used to modulate polymer hydration.

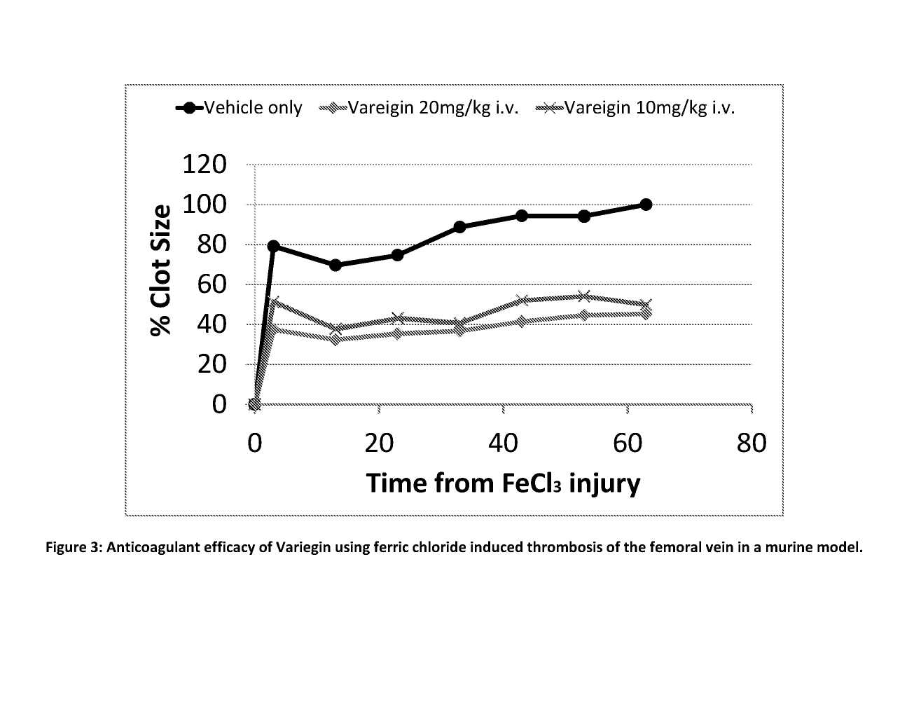

The solution provided by the present invention addresses the unmet clinical need of minimising the bleeding risk. This is supported by in vivo data. Dose response studies on administering variegin were carried out at increasing concentrations and a dose dependent inhibition of clot formation was observed using a murine ferric chloride (FeCl₃)-induced injury model of thrombosis, e.g. 10% (w/v) ferric chloride (FeCl₃)-induced thrombosis. At the highest dose employed 20 mg/kg a 55-60% inhibition of clot formation was observed with no prolongation of bleeding time (see FIG. 3).

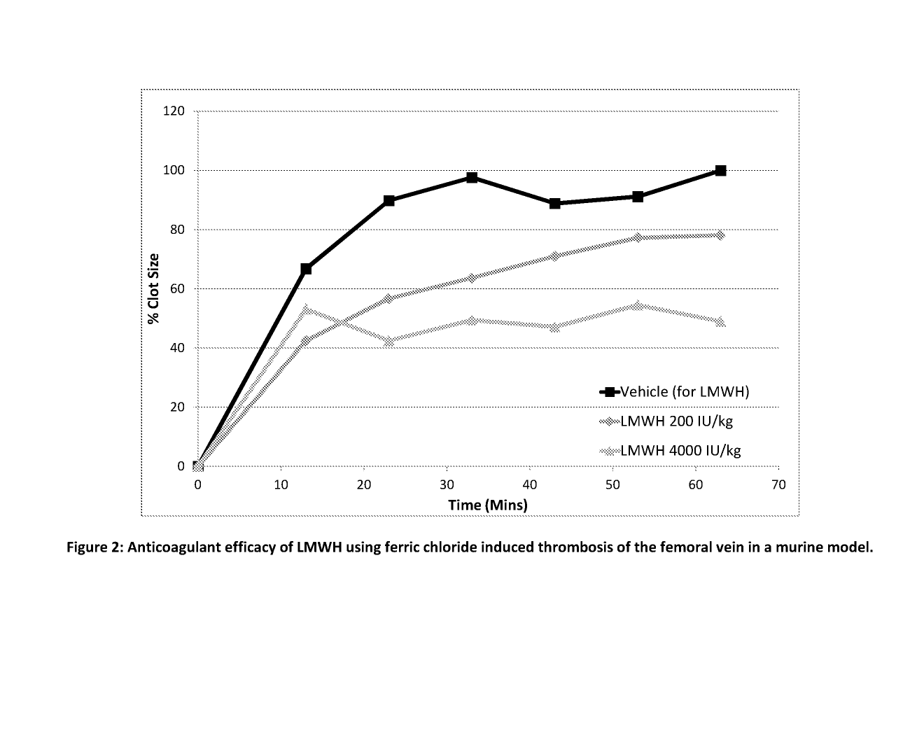

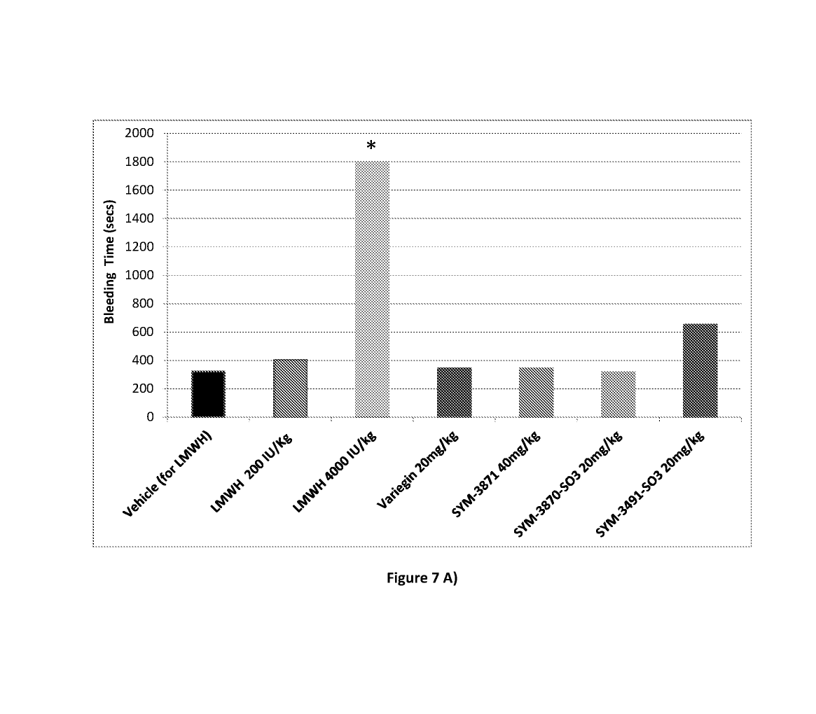

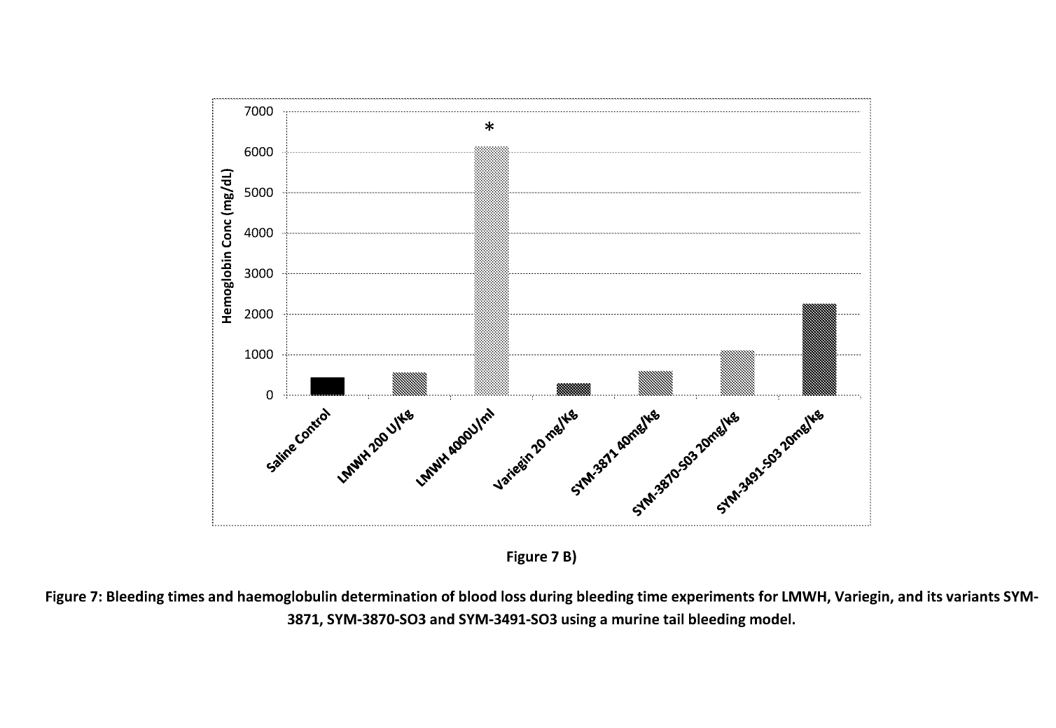

A derivative of heparin, fragmin, a low molecular weight heparin (LMWH), was used as a comparator. Generally, the therapeutic dose of fragmin administered to humans is 200 IU/kg. This same dose was administered intravenously to mice using the same FeCl₃ model of thrombosis as employed to test variegin. It was found that, at this dose, LMWH heparin was only able to reduce clot size by 29% with a small prolongation of bleeding time (average 403+/−74 seconds versus 334+/−36 seconds for controls); see FIG. 2 herein. More importantly, when the dose of LMWH was increased to 4,000 IU/kg, the LMWH was only able to inhibit clot size by 51%, yet this caused a prolongation of bleeding time to greater than 1,800 seconds (>5-fold) compared to control bleeding time and >11-fold blood loss. These data highlight the intricate balance that current anticoagulants need to achieve between being antithrombotic without causing bleeding. Strikingly, variegin shows superior efficacy at preventing clot formation: the highest dose employed, 20 mg/kg, showed 50-60% inhibition of clot size (n=4). Despite this remarkable anticoagulant effect, bleeding time and blood loss (measured by haemoglobulin lost during the bleeding time experiments) were similar to the controls (see FIGS. 7A & 7B herein). This is completely unprecedented as far as anticoagulants are concerned and it highlights the striking benefit variegin offers with respect to addressing the greatest unmet clinical need associated with all anticoagulant use: bleeding side effects.

In addition to low bleeding risk, variegin has several other features that provide an outstanding safety profile. For example, variegin may show low immunogenicity and toxicity. In size and structure, variegin is similar to bivalirudin which, unlike hirudin, is reported as not being immunogenic. However, although variegin is similar to bivalirudin, neither hirudin nor bivalirudin share the non-bleeding property of variegin. Furthermore, synthetic variegin is almost identical to natural variegin, a saliva peptide from a tick that takes at least 7 days of blood-feeding on cattle (and occasionally humans) to become fully engorged. Hence variegin has been subjected to strong evolutionary pressure to be both non-toxic and non-immunogenic, and also to be stable. Ex-vivo studies using human plasma indicate that variegin and its cleaved product have longer stability than bivalirudin, demonstrated by anticoagulant efficacy (Koh et al (2009). Non-competitive inhibitor of thrombin, ChemBioChem 10: 2155-2158). In a zebra fish model, variegin shows almost 3-fold greater efficacy at preventing the time to occlusion by thrombus compared to bivalirudin (Koh et al (2011) Crystal structure of thrombin in complex with s-variegin: insights of a novel mechanism of inhibition and design of tunable thrombin inhibitors, PLoS ONE 6(10): e26367).

Warfarin, despite its draw-backs, can be monitored in patients by performing a prothrombin clotting time (PT), where coagulation is triggered via the extrinsic pathway of coagulation by the addition of tissue factor. The time taken to form the clot measured by spectroscopy or mechanical methods is known as the prothrombin time. To normalize this clotting time, an International Normalised Ratio (INR) has been developed which essentially is the clotting time of the patient plasma divided by normal control plasma. The ratio derived can help to guide the dosage requirements for patients on warfarin, but in addition can give an indication if someone is over or under anticoagulated.

Whilst new oral anticoagulants claim that monitoring is not required, there is a concern that it may still be necessary to monitor the anticoagulant effect of these anticoagulants in certain circumstances, for example when a patient is bleeding.

The INR (International Normalized Ratio) is a measure of coagulation in a patient using the prothrombin time (PT) clotting method. In absence of anticoagulation therapy the INR is normally in the range of from 0.8 to 1.2. The target range for the INR in a patient being administered an anticoagulant, e.g. warfarin is from 2 to 3, but this is generally not achievable due to the bleeding risk. Other anticoagulants like rivaroxaban or apixaban or the like may require a 1.5 to 2-fold prolongation of activated partial thromboplastin time (aPPT). An advantage of variegin is that because of the lack of potential bleeding risk, dose escalation of administration can be achieved due to the necessity of not requiring the risk of bleeding to be balanced with anticoagulant efficacy. Therefore, it will be possible to monitor administration of variegin with the aPTT test beyond what is considered a typical therapeutic window. With variegin the therapeutic window might be safely widened, i.e. approaching the target of at least 2 to 3-fold prolongation of the aPPT, because, inter alia, variegin would be safer, due to the lack of bleeding risk.

The binding mode of variegin to thrombin has been identified by X-ray crystallography, confirming binding to both exosite 1 and the active site of thrombin (Koh et al (2011) Crystal structure of thrombin in complex with s-variegin: insights of a novel mechanism of inhibition and design of tunable thrombin inhibitors, PLoS ONE 6(10): e26367). Variegin is unique in that, inter alia, it combines both binding to exosite I and non-bleeding with direct and prolonged inhibition of the thrombin active site. Preliminary screening of variegin against other serine proteases and other key proteins of the coagulation systems, suggests variegin is very specific to thrombin (Koh et al (2007) Variegin, a novel fast and tight binding thrombin inhibitor from the tropical bont tick. Journal of Biological Chemistry 282: 29101-29113).

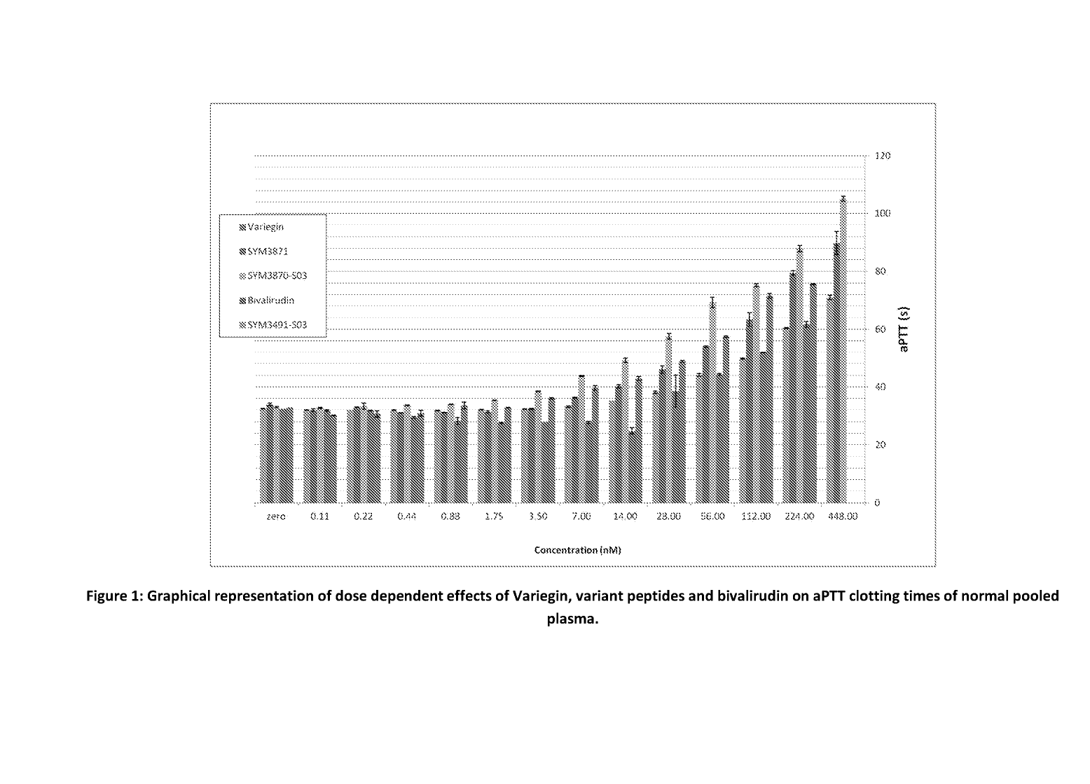

Variegin and bivalirudin show similar potency (IC50) in a purified system (Table 1, herein) which is also reflected in similar potency in normal platelet poor plasma using the aPTT assay (Table 2 and FIG. 1, herein). Fibrinogen binds to exosite 1 of thrombin and is therefore likely to compete with binding to variegin. Variegin is cleaved by thrombin to leave a smaller fragment that remains bound to the canyon-like cleft and exosite 1 of thrombin. This fragment does not inhibit small substrates like S-2238 yet inhibits the cleavage of fibrinogen to fibrin as would be expected by its interaction with exosite 1 (Koh et al (2009), Non-competitive inhibitor of thrombin, ChemBioChem 10: 2155-2158).

Taken together, all these data indicate that variegin is a superior anticoagulant, with improved efficacy and safety, compared to all current anticoagulants used in clinical practice because there appears to be no associated increased risk of bleeding with increased anticoagulant efficacy, whereas all current anticoagulants on the market possess a risk of bleeding, which makes dosing of those anticoagulants a fine balance between bleeding and anticoagulant properties. Variegin can achieve dose escalation with minimal risk of bleeding.

The invention will now be described by way of example only and with reference to the accompanying figures.

FIG. 1: Is a graphical representation of dose dependent effects of Variegin, variant peptides and bivalirudin on aPTT clotting times of normal pooled plasma. Increasing concentrations of variegin, variant peptides and bivalirudin were incubated with platelet poor plasma for 3 minutes prior to performing the aPTT according to the manufacturer's instructions. Data are presented as the mean±SEM.

FIG. 2: Is a graphical representation of anticoagulant efficacy of LMWH using ferric chloride induced thrombosis of the femoral vein in a murine model. Alexa-488 labelled fibrinogen was administered intravenously followed by therapeutic dose of LMWH (200 IU/kg), supratherapeutic dose (4000 IU/kg) or vehicle only (saline) administered intravenously prior to 10% (w/v) ferric chloride (FeCl₃)-induced thrombosis of the femoral vein. Clot size was monitored in real-time by monitoring the Alexa-488 over the area of FeCl₃ injury using Slidebook™ software for data acquisition and data analyses. All data have been expressed relative to the vehicle at the 63 minute point. Data are the average of a minimum of 2 replicates.

FIG. 3: Is a graphical representation of anticoagulant efficacy of Variegin using ferric chloride induced thrombosis of the femoral vein in a murine model. Alexa-488 labelled fibrinogen was administered intravenously followed by 10 mg/kg or 20 mg/kg of variegin or vehicle only (saline) administered intravenously prior to 10% (w/v) ferric chloride (FeCl₃)-induced thrombosis of the femoral vein. Clot size was monitored in real-time by monitoring the Alexa-488 over the area of FeCl₃ injury using Slidebook™ software for data acquisition and data analyses. All data have been expressed relative to the vehicle at the 63 minute point. Data are the average of a minimum of 4 replicates.

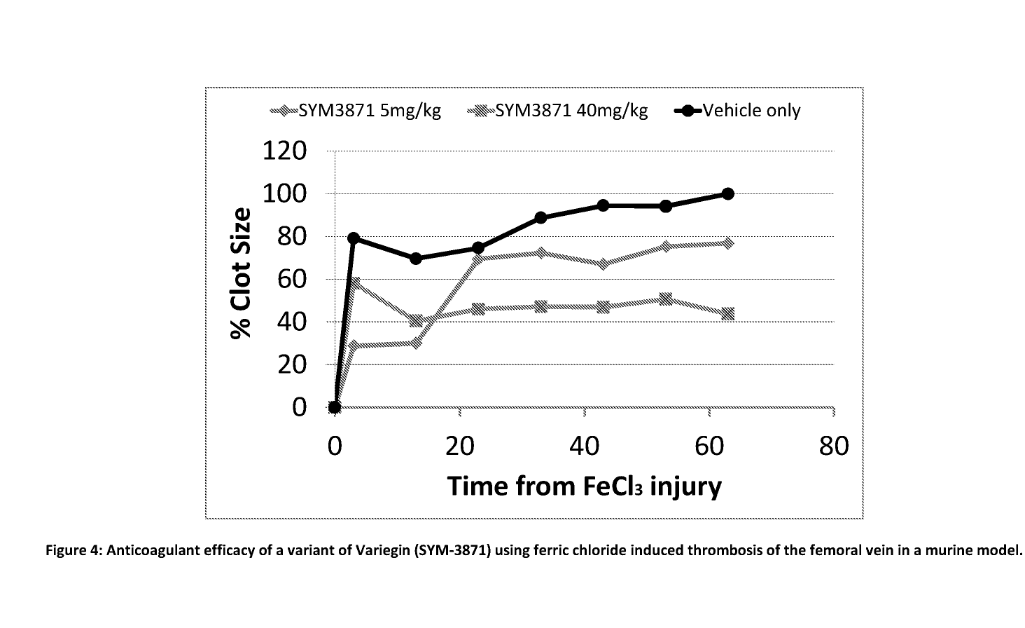

FIG. 4: Is a graphical representation of anticoagulant efficacy of a variant of Variegin (SYM-3871) using ferric chloride induced thrombosis of the femoral vein in a murine model. Alexa-488 labelled fibrinogen was administered intravenously followed by 5 mg/kg or 40 mg/kg of SYM-3871 or vehicle only (saline) administered intravenously prior to 10% (w/v) ferric chloride (FeCl₃)-induced thrombosis of the femoral vein. Clot size was monitored in real-time by monitoring the Alexa-488 over the area of FeCl₃ injury using Slidebook™ software for data acquisition and data analyses. All data have been expressed relative to the vehicle at the 63 minute point. Data are the average of a minimum of 2 replicates.

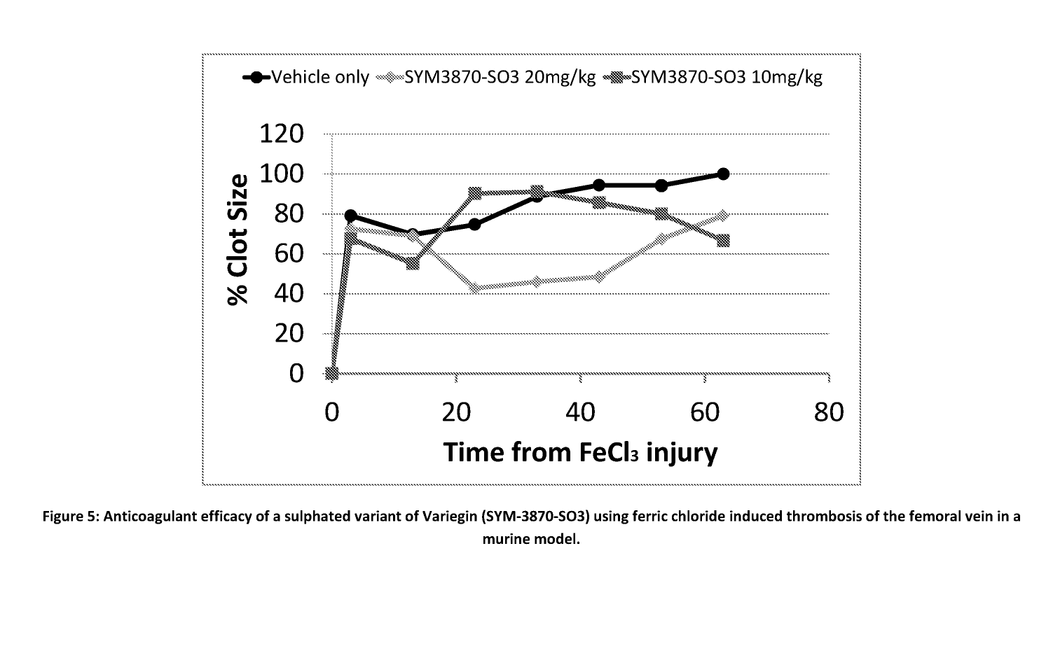

FIG. 5: Is a graphical representation of anticoagulant efficacy of a sulphated variant of Variegin (SYM-3870-SO3) using ferric chloride induced thrombosis of the femoral vein in a murine model. Alexa-488 labelled fibrinogen was administered intravenously followed by 10 mg/kg or 20 mg/kg of SYM-3870-SO3 or vehicle only (saline) administered intravenously prior to 10% (w/v) ferric chloride (FeCl₃)-induced thrombosis of the femoral vein. Clot size was monitored in real-time by monitoring the Alexa-488 over the area of FeCl₃ injury using Slidebook™ software for data acquisition and data analyses. All data have been expressed relative to the vehicle at the 63 minute point. Data are the average of a minimum of 1 replicate.

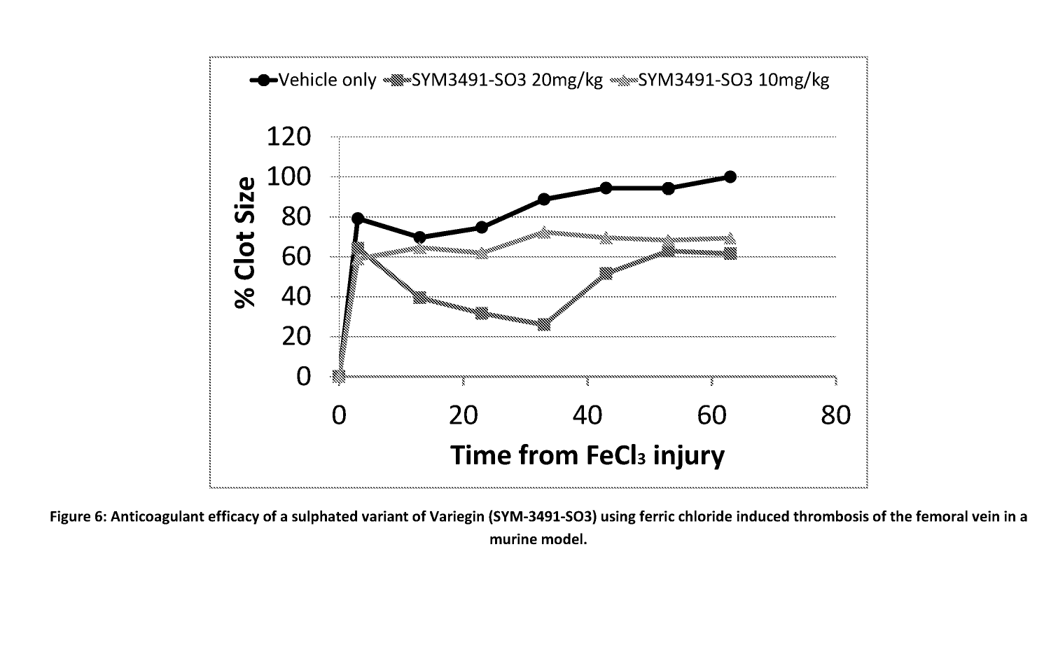

FIG. 6: Is a graphical representation of anticoagulant efficacy of a sulphated variant of Variegin (SYM-3491-SO3) using ferric chloride induced thrombosis of the femoral vein in a murine model. Alexa-488 labelled fibrinogen was administered intravenously followed by 10 mg/kg or 20 mg/kg of SYM-3491-SO3 or vehicle only (saline) administered intravenously prior to 10% (w/v) ferric chloride (FeCl₃)-induced thrombosis of the femoral vein. Clot size was monitored in real-time by monitoring the Alexa-488 over the area of FeCl₃ injury using Slidebook™ software for data acquisition and data analyses. All data have been expressed relative to the vehicle at the 63 minute point. Data are the average of a minimum of 1 replicate.

FIGS. 7 (A) & (B): Bleeding times and haemoglobulin determination of blood loss during bleeding time experiments for LMWH, Variegin, and its variants SYM-3871, SYM-3870-SO3 and SYM-3491-SO3 using a murine tail bleeding model. The same animals employed for anticoagulant efficacy were subjected to tail bleeding immediately following the end of the efficacy experiments. Tails were cut with a scalpel at 2 mm diameter using a home-made device for measuring tail diameter. The tails were immediately suspended into lml warmed saline and the time taken to cessation of bleeding monitored (panel A). The collected saline/blood mix at the end of the experiments were frozen and subjected to haemoglobin determination (panel B). On the FIGS. 7 (A) & (B) for the 4000 IU/kg dose of heparin indicated that the experiment was terminated for ethical reasons due to too much blood loss, these values are therefore underestimates of the true values. Doses of variegin and its variants were selected to yield at least 50% inhibition of fibrin formation during the experiment to enable a comparable bleeding risk potential at anticoagulant efficacy of ˜50% inhibition of fibrin formation with LMWH (4000 IU/kg). Therapeutic dose of LMWH (200 IU/kg) is also included within the data despite only 22% inhibition of fibrin clot formation at this dose.

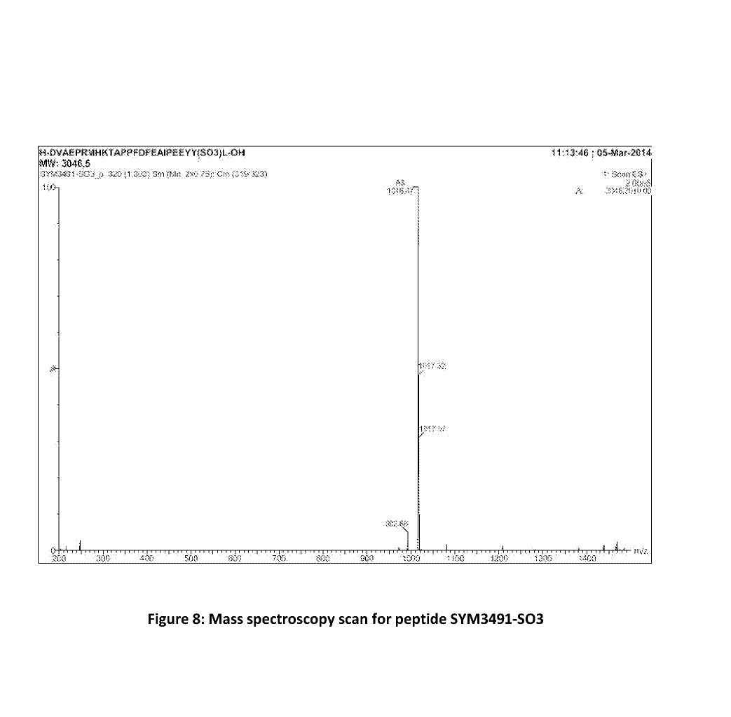

FIG. 8 is a mass spectroscopy scan for peptide SYM-3491-SO3.

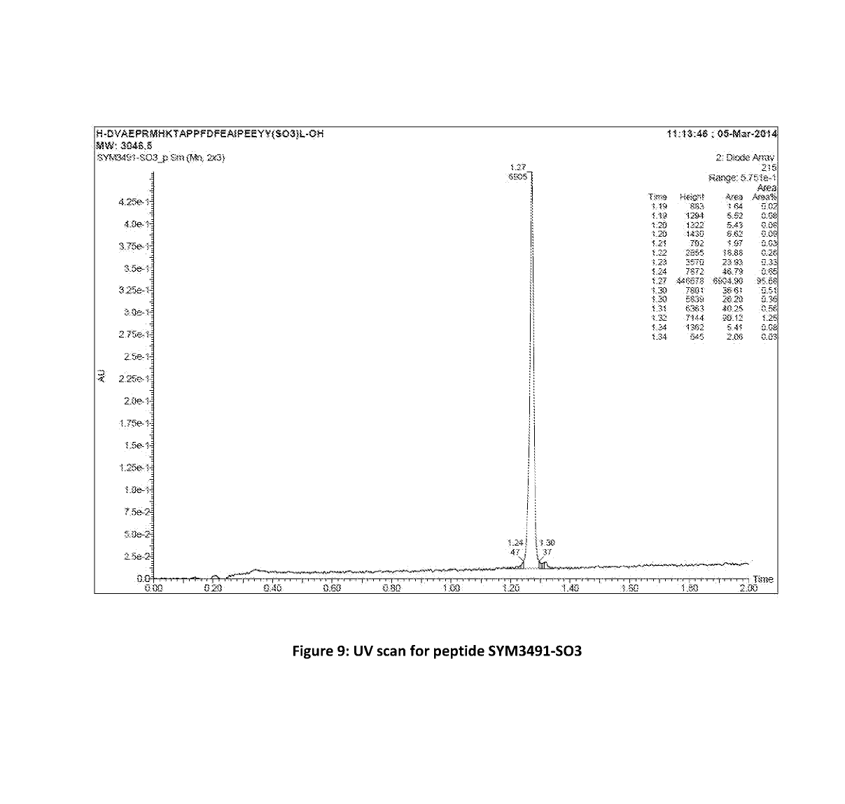

FIG. 9 is a UV scan for peptide SYM-3491-SO3.

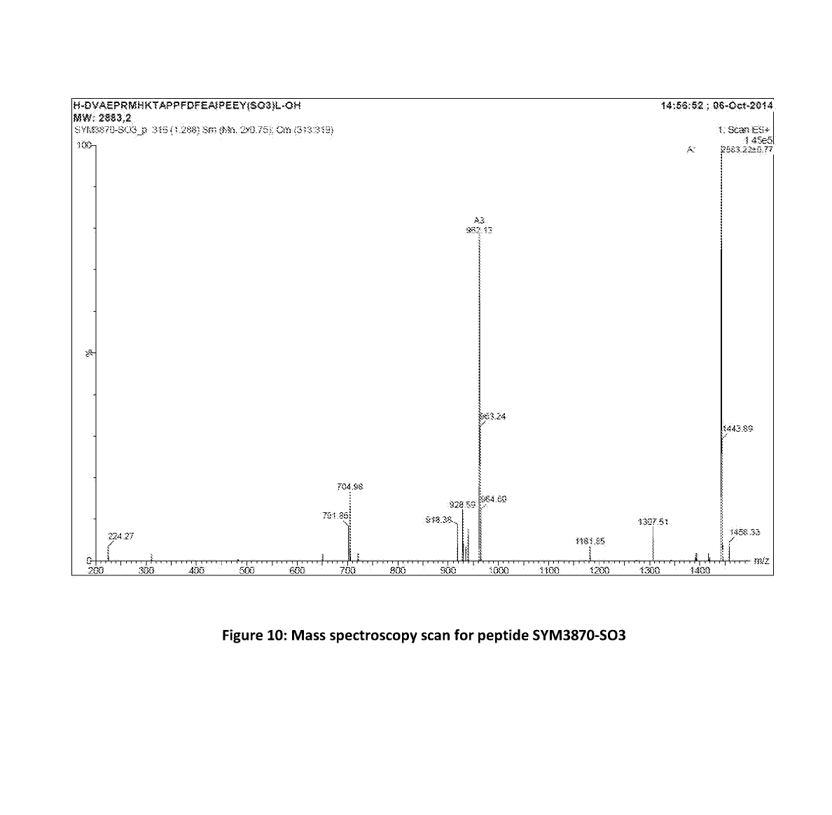

FIG. 10 is a mass spectroscopy scan for peptide SYM-3870-SO3.

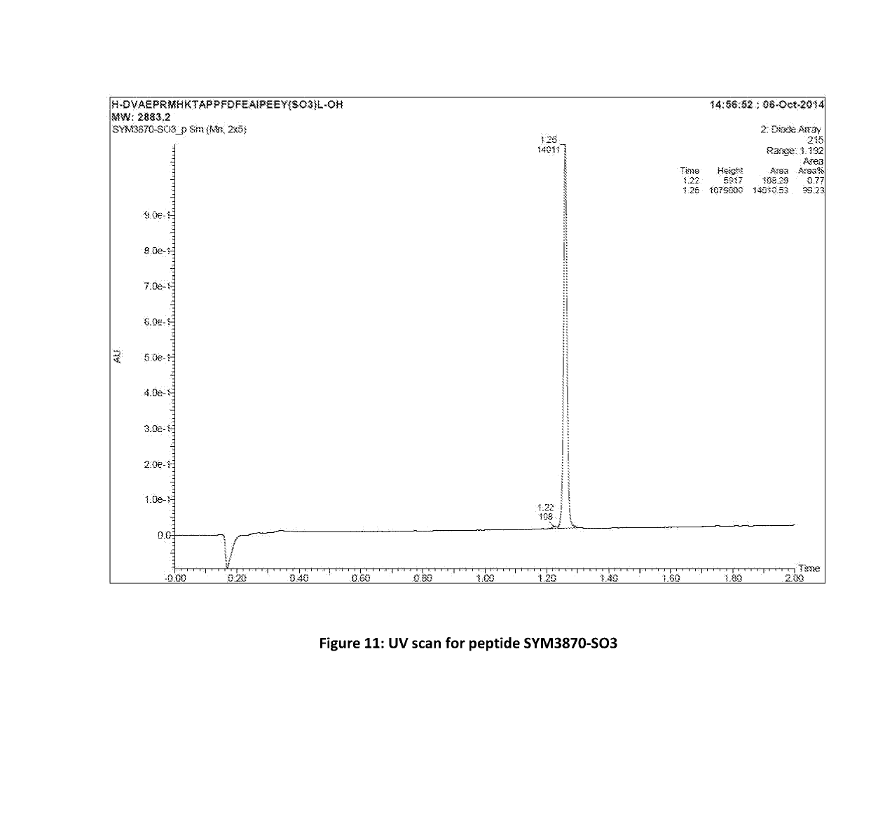

FIG. 11 is a UV scan for peptide SYM-3870-SO3.

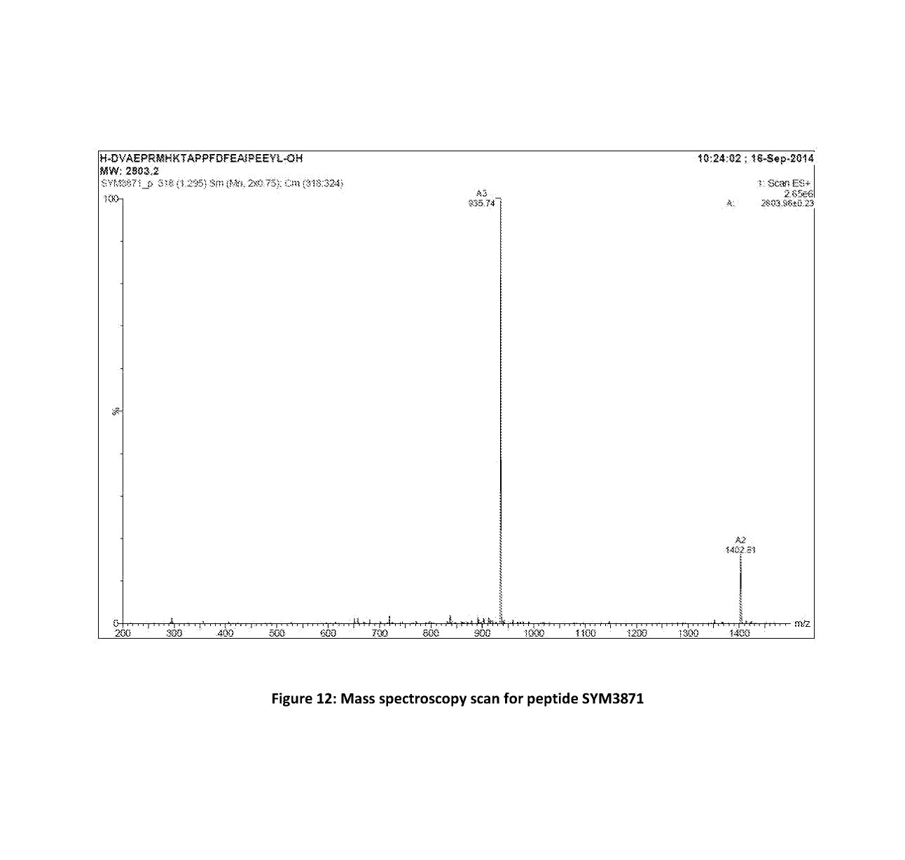

FIG. 12 is a mass spectroscopy scan for peptide SYM-3871.

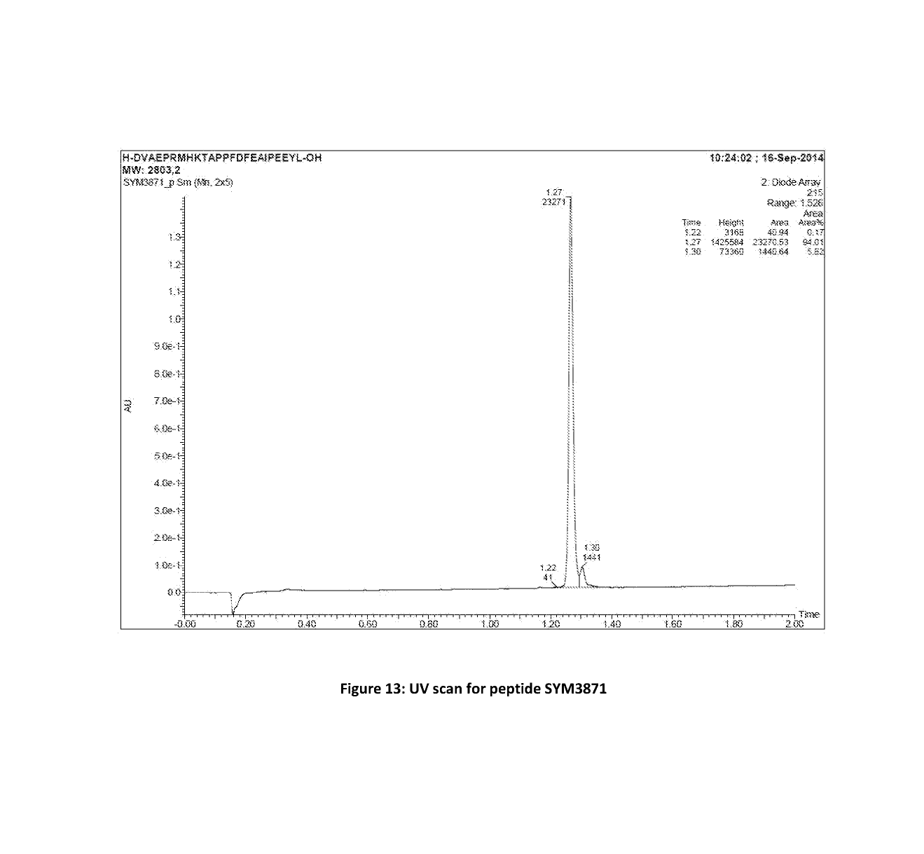

FIG. 13 is a UV scan for peptide SYM-3871.

### EXPERIMENTAL PROTOCOLS

Methods

Reagents

AlexaFluor488 conjugated fibrinogen was purchased from Invitrogen (Paisley, UK).

Animals

C57BL/6 male mice weighing between 20 and 30 g were used for all experiments. All procedures were approved by the University of Sheffield ethics committee and performed in accordance with the Home Office Animals (Scientific Procedures) Act 1985 of the United Kingdom.

Assessment of IC50 of Variegin and Variant Peptide Sequences

Thrombin activity was measured using a chromogenic substrate S-2238 (Quadratec). Various concentrations of variegin, peptide variants or bivalirudin were incubated with a final concentration of 2 nM of thrombin and incubated at 37° C. for 10 mins, prior to the addition of a final concentration of 100 μM S-2238 chromogenic substrate. Kinetic readings at 405 nm were monitored every 12 seconds for a total duration of 3 hours at 37° C. Gradients of initial rates were determined and employed to calculate IC₅₀ values using Grafit software. The results are provided in Table 1.

TABLE 1
IC50 data of Variegin, variant peptide and bivalirudin. Thrombin
activity was measured using a chromogenic substrate S-2238
(Quadratec). Various concentrations of variegin, peptide variants
or bivalirudin were incubated with a final concentration of 2 nM
of thrombin and incubated at 37° C. for 10 mins, prior to the
addition of a final concentration of 100 μM S-2238 chromogenic
substrate. The initial gradients were calculated to determine the
IC50 value. Data are presented as the mean ± SEM.

| Inhibitor | IC50 (nM) |
|---|---|
| Variegin | 72.0 ± 3.7 |
| SYM-3871 | 523.2 ± 23.3 |
| SYM-8370-S03 | 130.2 ± 16.5 |
| SYM-3491-S03 | 171.5 ± 6.5 |
| Bivalirudin | 79.3 ± 22 |

Blood Clotting Assays

Activated Partial Thromboplastin Time (aPTT)

The activated Partial Thromboplastin Time (aPTT) employed was the PTT Automate 5 reagent kit from Stago. The coagulometer (Start 4, Diagnostica Stago, Asnieres sur Seine Cedex, France) was preheated to 37° C. prior to starting any measurements. A sample of human normal pool platelet poor plasma (n=22) was defrosted at 37° C. from −80° C. 1.5 μL of increasing concentrations of variegin, variant peptides and bivalirudin to yield the desired final concentration were added with 50 μL of human plasma. Then 48.5 μL of PTT automate was added to the cuvette and left to incubate for 180 seconds at 37° C. in the coagulometer, after which 50 μL of 25 mM calcium chloride was immediately added to initiate clotting. At the end of the test, the time clotting times were recorded.

This method is known to the person skilled in the art. The results are provided in Table 2.

TABLE 2
Dose dependent effects of Variegin, variant peptides and bivalirudin on
aPTT clotting times of normal pooled plasma. Increasing concentrations of
variegin, variant peptides and bivalirudin were incubated with platelet poor plasma for
3 minutes prior to performing the aPTT according to the manufacturer's instructions.
Data are presented as the mean ± SEM.

| | Mean a PTT (s) | | | | |
|---|---|---|---|---|---|
| Concentration | | | SYM-3870- | | |
| (nM) | Variegin | SYM-3871 | S03 | Bivalirudin | SYM-3491-S03 |
| Zero | 32.97 ± 0.77 | 32.47 ± 0.30 | 33.70 ± 0.30 | 34.03 ± 0.44 | 32.53 ± 0.07 |
| 0.11 | 30.23 ± 1.11 | 31.88 ± 0.06 | 32.80 ± 0.23 | 32.03 ± 0.35 | 32.03 ± 0.07 |
| 0.22 | 30.73 ± 0.98 | 31.83 ± 0.28 | 33.43 ± 1.09 | 33.03 ± 0.15 | 32.10 ± 0.06 |
| 0.44 | 30.90 ± 1.16 | 29.50 ± 1.15 | 33.73 ± 0.03 | 31.07 ± 0.09 | 32.07 ± 0.03 |
| 0.88 | 33.57 ± 0.15 | 28.33 ± 0.19 | 34.10 ± 0.10 | 31.20 ± 0.06 | 31.83 ± 0.03 |
| 1.75 | 32.87 ± 0.20 | 27.57 ± 0.03 | 35.37 ± 0.09 | 31.47 ± 0.37 | 32.20 ± 0.15 |
| 3.50 | 36.07 ± 0.69 | 27.93 ± 0.38 | 38.53 ± 0.12 | 32.43 ± 0.13 | 32.33 ± 0.07 |
| 7.00 | 39.73 ± 0.62 | 27.73 ± 1.13 | 43.83 ± 0.12 | 36.30 ± 0.20 | 33.27 ± 0.27 |
| 14.00 | 42.97 ± 0.28 | 24.83 ± 5.56 | 49.20 ± 0.78 | 40.27 ± 0.60 | 35.33 ± 0.33 |
| 28.00 | 48.90 ± 0.25 | 38.60 ± 0.20 | 57.53 ± 1.03 | 46.13 ± 1.25 | 38.23 ± 0.48 |
| 56.00 | 57.37 ± 0.82 | 44.40 ± 0.10 | 69.33 ± 1.72 | 53.97 ± 0.26 | 44.17 ± 0.43 |
| 112.00 | 71.53 ± 0.18 | 51.93 ± 1.00 | 75.27 ± 0.49 | 63.43 ± 2.32 | 49.87 ± 0.20 |
| 224.00 | 75.55 ± 0.62 | 61.73 ± 0.58 | 87.90 ± 1.10 | 79.50 ± 0.75 | 60.33 ± 0.17 |
| 448.00 | No data | No data | 105.23 ± 0.84 | 89.77 ± 3.99 | 70.97 ± 0.69 |

The peptides used in the experimental work were as follows:

| Peptide- | | Purity | Weight | MW |
|---|---|---|---|---|
| code | Sequence | (%) | (mg) | (Da) |
| SYM-3491- | H-DVAEPRMHKT | 95.6 | 50.0 | 3046.4 |
| SO3 | APPFDFEAIPEE | | | |
| | YY(SO3)L-OH | | | |
| SYM-3870- | H-DVAEPRMHKT | 99.2 | 50.0 | 2883.2 |
| SO3 | APPFDFEAIPEE | | | |
| | Y(SO3)L-OH | | | |
| SYM-3871 | H-DVAEPRMHKT | 94.0 | 50.0 | 2803.2 |
| | APPFDFEAIPEE | | | |
| | YL-OH | | | |

The analytical data of the peptides is shown in FIGS. 8 to 13.

Intravital Microscopy for Real Time Assessment of Fibrin Formation In Vivo

Microscopic observation of thrombus formation following ferric chloride (FeCl₃) induced injury in vivo were made using an upright microscope (Nikon eclipse E600-FN, Nikon UK, Kingston upon Thames, United Kingdom) equipped for bright field and fluorescence microscopy and with a water immersion objective (40/0.80 W).

Mice were anaesthetised with an intraperitoneal (i.p.) injection of 125 mg/kg ketamine hydrochloride (Ketaset; Willows Francis Veterinary, Crawley, UK), 12.5 mg/kg xylazine hydrochloride (Bayer Suffolk, UK) and 0.025 mg/kg atropine sulphate (Phoenix Pharmaceuticals Ltd, UK). Cannulation of the trachea (to aid breathing) and carotid artery (for maintenance of anaesthesia and substance administration) were performed and the femoral vein was exposed. 100 μl of AlexaFluor488 conjugate fibrinogen (2 mg/ml) was administered via the carotid artery 5 min prior to application of a 3 mm×2 mm filter paper saturated with 10% (w/v) FeCl₃ being placed directly on the femoral vein for 3 minutes.

Real-time, Alexa488 nm (green channel) images using Slidebook imaging software (Version 5.0; Intelligent Imaging Innovations, 3i, Denver, USA) were taken to monitor thrombus formation in vivo at regular intervals for 1 h. The area was flushed with warm phosphate buffered saline (PBS) following FeCl₃ exposure and throughout the experiment.

Data Analyses Slidebook to determine Fibrin Clot Formation in Real Time

Real time images of thrombus formation were analysed using Slidebook image analysis software by setting a background region outside the thrombus area and measuring Alexa488 nm signal intensities above background. Setting individual background intensities for the green channel in this way allows selection of pixels that only show signal above background at each time frame. The resulting selection of pixels or “masked” region (defined as region used for data analyses) is then determined for the pixel's signal intensity for Alexa488 nm (encompassing intensity and area of signal). The Slidebook software allows for the calculation of background for each image file representing different time points in an automated manner, therefore allowing for background subtraction at each time point. Thrombus area is determined by quantifying pixel intensities above background (at each time point) in the Alexa488 nm channel. When establishing the background region, all time frames within the background are run as a movie to ensure that the region selected as background does not develop any clot growth over the duration of experiment. This is important for analyses with Slidebook because the same region of background is employed for signal determination at each time frame. Data generated is reflective of area intensity of each pixel and as background subtraction takes place with the same image/time frame this data provides an accurate assessment of Alexa488 area with intensity. Data is plotted as relative fluorescence units (RFU) over time. The percentage inhibition of clot formation is calculated relative to mice administered vehicle only for the 63 minute time point.

Bleeding Time Determinations

The same animals employed for anticoagulant efficacy were subjected to tail bleeding immediately following the end of the efficacy experiments. Tails were cut with a scalpel at 2 mm diameter using a home-made device for measuring tail diameter. The tails were immediately suspended into 1 ml warmed saline and the time taken to cessation of bleeding monitored.

Quantitative Assessment of Blood Loss

The collected saline/blood mix at the end of the experiments were frozen and subjected to haemoglobin determination. The amount of blood loss during the bleeding time experiments was determined by measuring haemoglobin concentration following red cell lysis using a kit for monitoring haemoglobin (Quantichrom haemoglobin assay kit, Bioassay Systems, Hayword, USA).

Description of Data

In a purified system the IC50 of variegin, and its variants were compared with bivalirudin for the ability to inhibit thrombin activity. Variegin showed comparable thrombin inhibition to bivalirudin, whereas the variants employed showed slightly less potency. In plasma, using the activated partial thromboplastin time (aPTT), similar potency was observed again between variegin and bivalirudin. SYM-3871 and SYM-3491-SO3 showed efficacy but slightly less than variegin and SYM-3870-SO3 showed a slight increase in potency compared to variegin.

Variegin and peptide variants were administered intravenously to monitor their anticoagulant effect using a murine ferric chloride-induced thrombosis model. Various doses were tested to approximately achieve 50% inhibition of fibrin clot formation during the course of the experiment. Bleeding time experiments were performed on the same animals immediately following the efficacy experiments. Low molecular weight heparin (LMWH) was employed as the comparator. At the therapeutic dose (200 IU/kg) of LMWH 22% inhibition of clot formation was achieved 63 minutes post ferric chloride injury, with a slight but not significant prolongation of average bleeding time from 334 to 403 seconds. In contrast at the dose of LMWH required to achieved 50% inhibition of fibrin clot formation a significant prolongation of bleeding time to >1800 seconds was observed, >5.5-fold prolongation. These experiments were terminated for ethical reasons, so the actual bleeding time is likely to be longer. Variegin, SYM-3871 and SYM-3871-SO3 at the dose required to achieve at least 50% inhibition of fibrin clot formation showed no significant prolongation of bleeding time, whereas SYM-3491-SO3 showed an approximate 2-fold prolongation of bleeding time. Similar trends to the bleeding time experiments were observed when haemoglobulin from the blood loss during these bleeding time experiments were measured. These data suggest that greater anticoagulant efficacy can be achieved for variegin and its variants compared with LWMH, which is limited by the amount of anticoagulant efficacy that can be achieved by increased risk of bleeding. In contrast, variegin, SYM-3871, SYM-3870-SO3 can achieve at least 50% inhibition of clot formation without any potential increased risk in bleeding. SYM-3491-SO3 increases risk of bleeding at increased anticoagulant efficacy but the benefit ratio still appears to be better than LMWH.

Whilst specific doses were administered for variegin and its variants these data have not taken into account the clearance of the peptides in vivo, it is therefore not possible to make direct comparisons on potency with these data between variegin and its variants. The key findings of these data are the efficacy: bleeding risk ratio which demonstrates that variegin and its variants are likely to be superior to LMWH.

1-51. (canceled)

52. A method of treating a patient suffering from an increased risk of thrombosis or preventing a subject developing a thrombotic event, said method comprising administering variegin to the patient in an amount of at least about 0.1 mg/kg (mass of drug compared to mass of patient) to provide ≧50% inhibition of thrombus formation without any potential risk of bleeding.

53. A method according to claim 52 in which the incidence of peri-operative or post-operative bleeding is substantially reduced.

54. A method according to claim 52 which comprises the post-operative administration of variegin to a patient.

55. A method according to claim 52 wherein the amount of variegin administered to a patient is sufficient to achieve a plasma concentration of variegin of from about 0.1 ng/ml to about 1.5 g/L.

56. A method according to claim 52 wherein the amount of variegin is sufficient to achieve a plasma concentration of at least 25 pM of variegin and is maintained for at least 2 hours in the patient.

57. A method according to claim 52 wherein the variegin is natural variegin.

58. A method according to claim 52 wherein the variegin is synthetic variegin.

59. (canceled)

60. A method according to claim 52 wherein the variegin is a variant of variegin.

61. A method according to claim 60 wherein the variant of variegin is one or more of SYM-3871, SYM-3870-SO3 and SYM-3491-SO3.

62. A method according to claim 60 wherein the variant of variegin is SYM-3871.

63. A method according to claim 60 wherein the variant of variegin is SYM-3870-SO3.

64. A method according to claim 60 wherein the variant of variegin is SYM-3491-SO3.

65. (canceled)

66. A method according to claim 52 which comprises the prophylactic use of variegin as an anticoagulant therapy.

67. A method according to claim 52 which comprises inducing anticoagulation in heparin-resistant patients.

68. A method according to claim 52 wherein the amount of variegin present is at least 10 mg/kg.

69. (canceled)

70. A method according to claim 52 wherein the variegin is administered parenterally, such as, intravenous, intramuscular or subcutaneous inj ecti on.

71. A method according to claim 52 wherein the variegin is administered enterally, such as, oral administration.

72. A method according to claim 52 wherein the variegin is in combination with a second therapeutically active agent.

73. A method according to claim 72 wherein the second therapeutically active agent is an antithrombotic agent or a fibrinolytic agent.

74. A method according to claim 52 wherein the patients are those requiring dose escalation e.g. obese, kidney diseases, liver disease, iv drug users, pregnancy, paediatric, geriatric, cancer, extended-duration prophylaxis of venous thromboembolism (VTE) in acute medically ill patients, breastfeeding, percutaneous coronary intervention, orthopaedic surgery, abdominal aortic aneurysm (AAA); those patients requiring additional platelet inhibition with anticoagulation, such as, percutaneous coronary intervention (PCI); patients on thrombolytic therapy (tissue plasminogen activator, (tPA)) for treatment of ischaemic stroke; or patients requiring cardiopulmonary bypass surgery or extracorporeal membrane oxygenation (ECMO) and dialysis.

75-80. (canceled)
